(19) United States
(12) Patent Application Publication
(10) Pub. No.: US 2024/0253037 A1
(43) Pub. Date: Aug. 1, 2024

(54) MICROFLUIDIC CHIP FOR ATTRACTING AND TRAPPING A SPECIFIC BIOLOGICAL ELEMENT

(71) Applicants: UNIVERSITE DE MONTPELLIER (UM), Montpellier (FR); CENTRE NATIONAL DE LA RECHERCHE SCIENTIFIQUE (CNRS), Paris (FR); ECOLE NATIONALE SUPERIEURE DE CHIMIE DE MONTPELLIER (ENSCM), Montpellier (FR)

(72) Inventors: Jean-Marie RAMIREZ, Usclas-du-Bosc (FR); Benoît CHARLOT, Montpellier (FR); Sébastien MEANCE, Montpellier (FR); Xavier GARRIC, Montpellier (FR); Coline PINESE, Montpellier (FR); Isabelle GUIRAUD, Saint Hilaire de Brethmas (FR)

(73) Assignees: UNIVERSITE DE MONTPELLIER (UM), Montpellier (FR); CENTRE NATIONAL DE LA RECHERCHE SCIENTIFIQUE (CNRS), Paris (FR); ECOLE NATIONALE SUPERIEURE DE CHIMIE DE MONTPELLIER (ENSCM), Montpellier (FR)

(21) Appl. No.: 18/560,483

(22) PCT Filed: May 17, 2022
(86) PCT No.: PCT/FR2022/050938

§ 371 Date: Nov. 13, 2023

(30) Foreign Application Priority Data
May 17, 2021 (FR) FR2105106

(51) Int. Cl. B01L 3/00

(52) U.S. Cl. CPC B01L 3/502753; B01L 2300/0861; B01L 2400/086

(57) ABSTRACT

The present invention relates to a microfluidic chip capable of attracting and trapping a specific biological element, said chip comprising: a reservoir (1) consisting of a matrix comprising a chemoattractant compound capable of attracting the biological element, and at least one array of microchannels (2) arranged between the reservoir (1) and the external medium (3) of the chip and enabling the chemoattractant compound to pass towards said medium and the biological element present in said medium to pass towards the reservoir (1), characterized in that each microchannel is in the form of a harpoon comprising at least two arrows spaced apart longitudinally and directed towards the reservoir, in which each arrow comprises two branches each having a free end forming an acute angle of between 10° and 80°, and in which each arrow comprises two openings (7) communicating with a longitudinal part of the microchannel and having a width of between 5 μm and 30 μm.

### TECHNICAL FIELD

The present application relates to the field of microfluidic devices capable of attracting and trapping a specific biological element. More precisely, the present application concerns a microfluidic chip capable of attracting and trapping in vivo a specific biological element, such as a prokaryotic or eukaryotic cell.

### PRIOR ART

Every year in France, some 400,000 new cases of cancer are diagnosed, and around 150,000 deaths.

There are different types of cancer, varying according to the tissue in which they develop. A distinction can be made between solid tumors, which are characterized by a localized mass of cancer cells, and blood cell tumors, which are diffuse, with cancer cells circulating in the bone marrow or blood. Blood cell tumors, also known as hematopoietic cancers, are cancers affecting the blood or lymphoid organs, such as leukemia and lymphoma.

Solid tumors include carcinomas and adenocarcinomas, cancers arising from epithelial tissue. A further distinction is made between sarcomas, which correspond to cancer cells arising in a so-called supporting tissue: osteosarcoma for bone, liposarcoma for fat, and myosarcoma for muscle.

For both men and women, solid cancers represent 90% of all cancers, of which prostate, lung and colorectal cancers are the three most common in men, and breast, colorectal and lung cancers the three most common in women. Although cancer mortality fell by 1.5% a year in men and 1% a year in women between 1980 and 2012 (standardized rates), there is still a need for effective solutions to treat and prevent these solid tumors and their complications.

To date, surgery is one of the main treatments for solid tumors, involving resection of the entire tumor whenever possible, and possibly the tissue surrounding the tumor, known as the resection margin. Surgery may be used as the sole treatment when the tumor is very localized, particularly when the tumor is at an early stage, but it is often combined with other treatments such as radiotherapy, which is also a local treatment, and/or medical treatments such as chemotherapy, which is a systemic treatment potentially acting on all cancer cells in the body.

The advantage of local surgery for the treatment of solid tumors is the possibility of removing the entire tumor when possible, and preserving organs and anatomical structures not affected by cancer cells. It also limits the side effects of radiotherapy treatment, such as burns or the generation of radiation-induced cancers, and of chemotherapy, such as skin reactions, nausea, vomiting, diarrhea, muscle pain, fatigue, hair loss and chemo-induced cancers.

To reduce the relapse rate, the resection zone includes a healthy tissue zone around the tumor, which corresponds to the resection margin. At this stage, several scenarios need to be considered. In the first case, the tumor cells of the primary tumor are included in the resection zone, and there will be no recurrence. In a second case, certain tumor cells are located beyond the resection zone, either locally or at a distance, and a recurrence is then possible, either locally or at a distance, to form metastases, i.e., secondary colonies of cancer cells which spread at a distance from the organ affected by the initial tumor, causing a so-called “metastatic” cancer in an organ other than the one in which the solid tumor was located.

It is thus essential to prevent the risk of local recurrence and metastatic cancer after resection of a solid tumor.

Chemotherapy can be used after local surgery to resect the tumor, known as “adjuvant chemotherapy”, to prevent recurrence and/or metastatic cancer. However, as previously mentioned, this drug treatment is associated with a wide range of side effects. Among the most significant side effects associated with the use of chemotherapy are chemo-induced cancers, which by definition correspond to new tumors arising in patients treated with cytotoxic drugs for a first malignancy and caused by these drugs, and the risk of generating cancer cell resistance to chemotherapeutic treatment, thus severely limiting the possibilities of eliminating said cells.

There is a need for a solution that effectively eliminates remaining cancer cells after resection of a solid tumor, in order to prevent and/or reduce the risk of local recurrence and/or development of metastatic cancer, and that is effective and free of short- and medium-term drug side effects.

Microfluidic devices have been described in the prior art for use in cancer treatment. In particular, reference can be made to document WO2018/089989 A1, which describes an ex vivo device for treating cancer by subjecting a biological fluid such as blood to electromagnetic radiation specific to the type of cancer cell targeted and capable of destroying it.

Reference may also be made to US2018111124 A1, which describes a microfluidic device comprising one or more microfluidic channels and one or more wireless bipolar electrode arrays enabling high-throughput capture of circulating tumor cells in a conductive ionic solution, by applying a 40 KHz alternating electric field to a biological sample. Cells captured in this way can be used to diagnose cancer or evaluate the effect of anti-cancer treatment. No prior art document describes or suggests a microfluidic chip for attracting and destroying, preferably in vivo, a biological element and in particular a cancer cell after resection of a solid tumor and thus preventing and/or reducing the risks of local recurrence and/or metastatic cancer development, which is simple and inexpensive to manufacture, effective and easy to implement in vivo for clinical application.

To meet this need, the present invention proposes a microfluidic chip capable of attracting, trapping and ultimately destroying a specific biological element, comprising a reservoir consisting of a matrix comprising a chemoattractant compound capable of attracting the biological element, at least one array of microchannels arranged between the reservoir and the medium outside the chip and allowing the chemoattractant compound to pass towards said medium and the biological element present in said medium to pass towards the reservoir, characterized in that each microchannel is in the form of a harpoon comprising at least two arrows spaced longitudinally from one another and directed towards the reservoir, wherein each arrow comprises two branches each having a free end forming an acute angle of between 10° and 80°, and wherein each arrow comprises two openings communicating with a longitudinal part of the microchannel and having a width of between 5 μm and 30 μm.

### DISCLOSURE OF THE INVENTION

The present invention relates to a microfluidic chip capable of attracting and trapping a specific biological element, said chip comprising:
a reservoir (1) consisting of a matrix comprising a chemoattractant compound capable of attracting the biological element
at least one array of microchannels (2) arranged between the reservoir (1) and the external medium (3) of the chip and enabling the chemoattractant compound to pass towards said medium and the biological element present in said medium to pass towards the reservoir (1), characterized in that each microchannel is in the form of a harpoon comprising at least two arrows spaced apart longitudinally and directed towards the reservoir, in which each arrow comprises two branches each having a free end forming an acute angle of between 10° and 80°, and in which each arrow comprises two openings communicating with a longitudinal part of the microchannel and having a width of between 5 μm and 30 μm.

### BRIEF DESCRIPTION OF THE DRAWINGS

FIG. 1 shows an array of microchannels (2) forming part of the chip according to the invention.

FIG. 2 shows an electron microscope view (X414 magnification) of an array of microchannels (2) forming part of the chip according to the invention. A distinction is made between cells (15) trapped within the chip and cells (16) present in the external environment.

FIG. 3 is a cross-sectional view of the chip according to the invention.

FIG. 4 shows the upper part (9) of the chip seen from above.

FIG. 5 shows the lower part (8) of the chip seen from above.

FIG. 6 shows an exploded view of the underside of the chip according to the invention.

FIG. 7 shows an exploded view of the top of the chip according to the invention.

FIG. 8 shows a cross-sectional view of the chip according to the invention at a scale of 5:0.5 cm.

FIG. 9 shows a cross-sectional view of the chip at a scale of 5:1 cm.

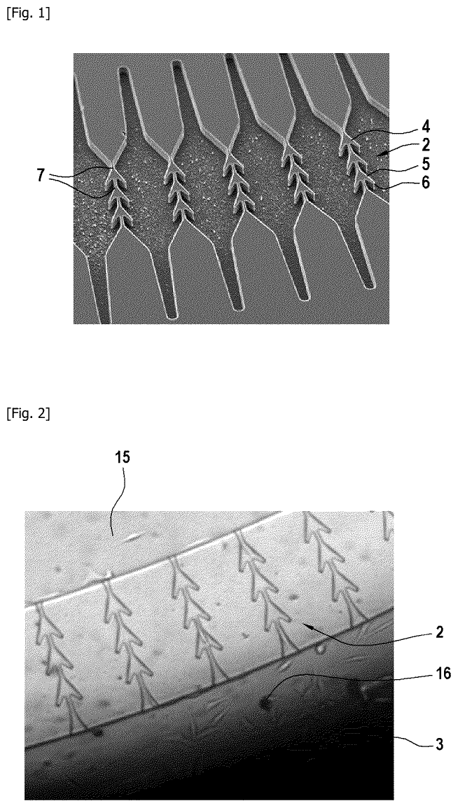

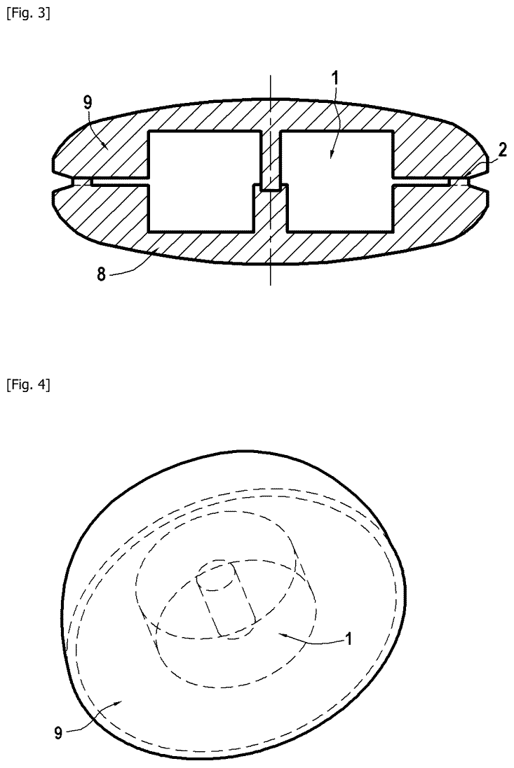

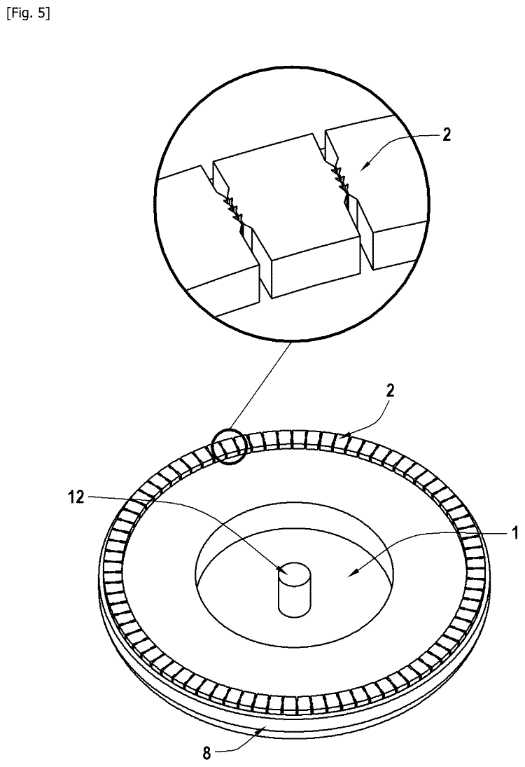

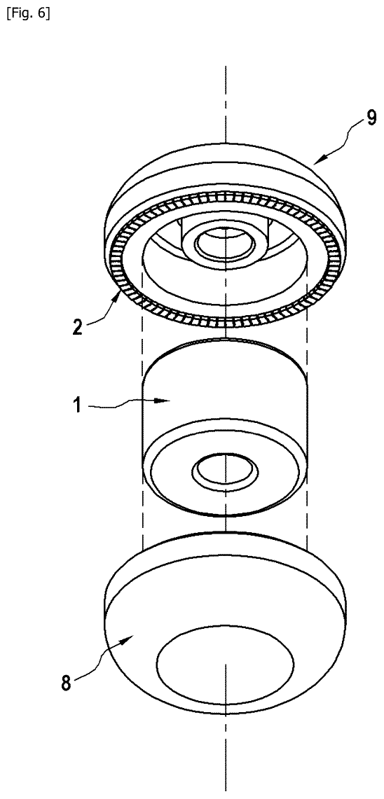

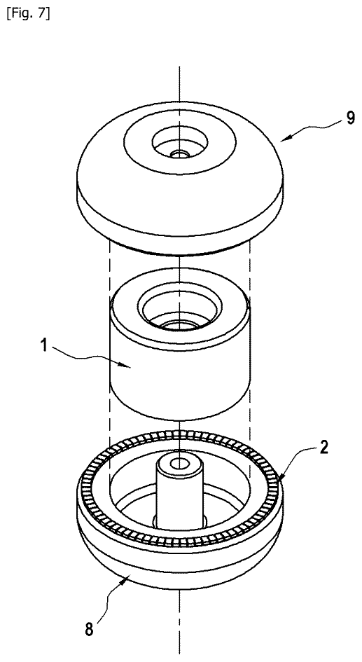

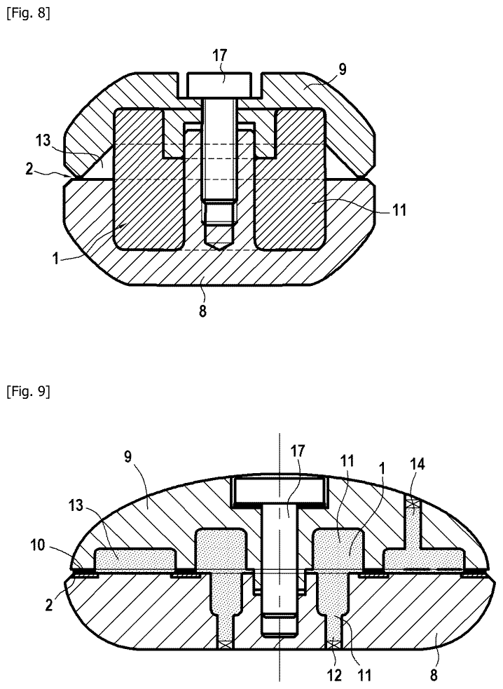

### DETAILED DESCRIPTION

The Applicants have developed a microfluidic chip for attracting and trapping a biological element, such as a prokaryotic or eukaryotic cell. This microfluidic chip is particularly interesting for attracting and trapping a cancer cell in vivo after resection of a solid tumor in order to prevent and/or reduce the risk of local recurrence and/or development of metastatic cancer. The microfluidic chip developed by the Applicants is particularly advantageous because it can attract, trap and ultimately destroy a biological element without the need for one or more electrodes. In particular, the absence of electrodes makes it easier to manufacture the microfluidic chip, to limit production costs and also to facilitate the implementation of the microfluidic chip in vivo. Indeed, the particular structure of the microchannels used in the microchip according to the invention enables the passage of a biological element within said chip, from the external environment towards the reservoir consisting of a matrix comprising a chemoattractant compound capable of attracting the biological element, while preventing said biological element from exiting the chip towards the external environment. In this way, the chip according to the present invention not only attracts a biological element present in the chip's external environment, but also traps it within said chip and ultimately destroys it, since the biological element deprived of the resources necessary for its survival ends up being destroyed.

### Definitions

A “microfluidic chip” is a device comprising an array of microchannels, i.e., channels of micrometric size, etched or molded in a material, connected to each other and linking the inside of the chip to the outside of the chip by means of inlets and outlets drilled through the chip, to perform a desired function. The microfluidic chip can be obtained by specific processes such as deposition and electroplating, etching, bonding, injection molding, embossing, soft lithography, anodic soldering, or any other technologies. These manufacturing processes are known to those skilled in the art. In the context of the present invention, the intended function of the microfluidic chip is to be able to attract in vivo a specific biological element, preferably a eukaryotic cell in particular a cancer cell, and to destroy it in vivo.

The “array of microchannels” corresponds to a multitude of channels, connected to the outside of the chip by inlets and outlets drilled through the chip. Microchannels can be manufactured from a mold, for example, or directly in the microfluidic chip material. The number of microchannels varies according to the diameter of the chip, the width of the microchannels and the spacing between them.

Each microchannel making up the array of microchannels corresponds to a passageway whose height can be from a few micrometers to a few hundred micrometers, with a length from a few hundred micrometers to a few millimeters. The width of a microchannel corresponds to the horizontal distance between the two points on opposite edges of the cross-section that are furthest apart. The height of a microchannel corresponds to the vertical distance between the points on opposite edges of the cross-section that are furthest apart. The length of a microchannel is the distance between the two ends of said channel, the length of a microchannel corresponding to the longest dimension. The two shorter dimensions generally define the aforementioned cross-section. By “harpoon-shaped microchannel” in the sense of the present invention, we refer to the fact that each microchannel forming part of the array of microchannels has a spear-like longitudinal shape.

The microfluidic chip is preferably “designed to be implanted in vivo”, i.e., the microfluidic chip is designed and suitable for implantation in a living being, preferably a mammal and in particular a human being. More specifically, this means that the chip can be implanted in a living being without interfering with or degrading the tissues with which it is in contact, and that said chip is able to function in vivo, i.e., to attract, trap and destroy a specific biological element in vivo, preferably a eukaryotic cell such as a cancer cell.

For the purposes of this invention, “biological element” refers to any element comprising genetic information in the form of RNA or DNA and likely to be found within a living organism, i.e., in vivo, such as prokaryotic cells, eukaryotic cells and microorganisms. Possible biological elements include a prokaryotic cell such as a bacterium, or a eukaryotic cell such as an animal cell.

The term “specific biological element” or “target biological element” refers to the biological element of interest in the context of the chip's use, i.e., the biological element to be attracted and destroyed in vivo. The chemoattractant compound present in the reservoir is chosen to attract the biological element of interest when using the microfluidic chip.

Preferably in the context of the invention, the biological element of interest is a prokaryotic or eukaryotic cell, even more preferably the biological element of interest is a eukaryotic cell. Even more preferably, the eukaryotic cell is a cancer cell, preferably a metastatic cancer cell.

A “cancer cell” is a cell in which one or more major DNA lesions have occurred, transforming the normal cell into a cancer cell capable of proliferating to form a group of identical transformed cells, i.e., a tumor. In particular, a cancer cell is referred to when the cell in question displays a number of characteristics, such as independence from cell growth regulation signals, an ability to escape the process of programmed cell death, and a capacity to divide indefinitely.

More specifically, in the context of the present invention, “cancer cell” refers to a cell originating from an initial or primary solid tumor, present at or near the solid tumor resection zone.

Preferably in the context of the present invention, “cancer cell” also refers to a “metastatic cancer cell”, i.e., a cancer cell capable of or having migrated through the body via blood or lymphatic vessels from the original tumor and capable of or having colonized one or more other tissues close to or at a distance from said tumor, thus forming metastases, at the origin of “metastatic cancers” or “metastatic tumors”. In this context, the cancer cell is a cell originating from a so-called secondary or tertiary solid tumor, corresponding to metastatic tumors in a second or third tissue or organ other than that of the initial tumor.

For the sake of clarity, reference is made to “specific biological element”, “a prokaryotic cell”, “a eukaryotic cell”, “a cancer cell”, “a metastatic cell” in the singular, it being understood that the microfluidic chip according to the present invention is capable of attracting and destroying several of said elements and cells in vivo. It is also worth noting that the microfluidic chip according to the present invention is capable of attracting and destroying in vivo several specific biological elements which may be of different natures, said elements being attracted within the chip by the specific choice of chemoattractant compound(s) present in the reservoir.

In the context of the present invention, the term “prevent” means to reduce the risk of acquiring a specified disease or disorder, or to reduce or slow down the onset of symptoms of that disease. For example, in the context of the present invention, the term “prevent” may refer to the reduction of the risk of propagation of an infection when the biological element is a prokaryotic cell, or to the reduction of the risk of local recurrence of cancer and/or the risk of the appearance of metastases, more specifically metastatic cancers, when the biological element is a eukaryotic cell of the cancerous type.

In the context of the present invention, the term “treat” refers to an improvement or reversal of a specified disease or disorder, or of at least one discernible symptom. The term “treat” may also refer to reducing or slowing the progression of the disease or disorder, or the onset of symptoms of that disease or disorder. For example, in the context of the present invention, the term “treat” may correspond to reducing or slowing down the progression of an infection when the biological element is a prokaryotic cell, or to reducing or slowing down the appearance of metastases, more precisely metastatic cancers, when the biological element is a eukaryotic cell of the cancer type.

For the purposes of the present invention, the microfluidic chip is preferably intended to be implanted in vivo in a subject.

The subject in the context of the present invention is a living being, preferably a mammal and more particularly human beings, children, men or women.

The term “solid cancer” or “solid tumor” refers to an individualized mass of cancer cells in any tissue such as skin, mucosa, bone, or any other tissue present in organs, i.e., carcinomas or endocarcinomas originating from epithelial cells such as skin, mucosa, glands and sarcomas originating from cells of connective and supporting tissues such as bone, cartilage.

Preferably, in the context of the present invention, “solid cancer” or “solid tumor” refers to a carcinoma such as cancer of the breast, lung, prostate, bladder, salivary glands, skin, intestine, colon-rectum, thyroid, cervix, endometrium and ovaries, lip-mouth-larynx cancer, kidney, liver, brain, testicles, pancreas, preferably breast cancer. These examples of solid tumors are not exhaustive.

The term “chemoattractant compound” refers to any compound capable of chemotactically attracting a biological element, preferably a cell expressing membrane receptors specific to this compound on its surface, said biological element moving as a function of the concentration gradient of the chemoattractant compound. In the context of the present invention, the chemoattractant compound is able to induce the displacement of one or more specific biological elements as a function of the concentration gradient of said compound by positive chemotaxis, the biological element moving towards the region where the concentration of chemoattractant compound is highest.

Particularly in the context of the present invention, the chemoattractant compound is said to be “able to attract” a specific biological element when it enables the movement of said element within the microfluidic chip and in particular towards the chemoattractant compound reservoir in which the highest concentration of chemoattractant compound is found. The person skilled in the art will know how to characterize the specific biological element of interest in order to choose the chemoattractant compound suitable for attracting said element to the chip.

In the context of the present invention, the chemoattractant compound is chosen as a function of the specific biological element of interest.

When the biological element is a eukaryotic cell, the chemoattractant compound is chosen according to the type of membrane receptors this cell expresses.

More specifically, when the biological element is a eukaryotic cell, the chemoattractant compound can be a cytokine, i.e. a soluble polypeptide or protein synthesized by a cell and acting remotely on other cells to regulate their activity and function via membrane receptors, chosen from chemokines, granulocyte and macrophage colony-stimulating factors such as M-CSF, G-CSF, CSF-1, growth factors and transforming growth factors such as TGF alpha, TGF beta, EGF, betacellulin, amphiregulin, heregulin, HBEGF, FGF, VEGF, tumor necrosis factors such as NGF, TNF alpha, TNF beta, interferons such as IFN alpha, IFN beta, IFN gamma, IFN lambda and interleukins such as IL-1 to IL-38.

Preferably when the biological element is a eukaryotic cell such as a cancer cell, the chemoattractant compound is selected from chemokines, growth factors and transforming growth factors.

A chemokine is a small protein (8 to 14 kilodaltons) characterized by the presence of four cysteine residues in conserved positions, enabling the formation of its three-dimensional structure. Chemokines can be classified into four subfamilies, depending on the spacing between two of their N-terminal cysteines: the CXC or alpha family, whose first two cysteines are separated by any amino acid; the CC or beta family; the CX3C or delta family; and the C or gamma family. The chemokine in the context of the present invention may, for example, be selected from the following chemokines: CXCL12, also known as stromal cell-derived factor 1 (SDF-1), CCL5, CCL2, CCL3, CCL7, CCL19, CCL21, CCL22, CCL25, CXCL1, CXCL5, CXCL6, CXCL8, CX3CL1.

A growth factor is a low molecular weight protein (less than 30 kilodaltons) which stimulates cell multiplication and is recognized by specific membrane receptors, most often tyrosine kinases. The growth factor in the context of the present invention may, for example, be chosen from TGF alpha or beta (transforming growth factor alpha or beta), FGF (fibroblast growth factor alpha), EGF (epidermal growth factor), betacellulin, amphiregulin, heregulin, HBEGF, VEGF (vascular endothelial growth factor), PDGF (platelet-derivative growth factor).

When the biological element is a prokaryotic cell, such as a bacterium, the chemoattractant compound can be a peptide bearing a formylated N group, such as N-formylmethionyl-leucyl-phenylalanine (FMLP), or carbohydrate molecules such as glucose.

In the context of the present invention, the chemoattractant compound is comprised in a matrix which is composed of a biocompatible material as defined herein. The biocompatible material of the matrix is chosen specifically according to the chemoattractant compound, the desired release profile and the context of use of the microfluidic chip.

In the context of the present invention, “external environment” refers to the tissue around the microfluidic chip when implanted in vivo, and more specifically to the tissue directly in contact with the chip, up to 300 mm, preferably up to 150 mm, and even more preferably up to 100 mm around the chip.

By “resection of a solid tumor” we mean the removal, ablation or excision of a solid tumor, for example by surgery.

The present invention concerns a microfluidic chip capable of attracting and trapping a specific biological element in vivo. Advantageously, the chip according to the invention enables passive destruction of the biological element trapped within the chip, which, deprived of the conditions necessary for its survival, ends up being destroyed.

The numerical references used in this detailed description of the invention refer to the figures in this application, which are intended to illustrate the invention, but are not limited to it.

A first object of the invention relates to a microfluidic chip capable of attracting and trapping a specific biological element, said chip comprising:
a reservoir (1) consisting of a matrix comprising a chemoattractant compound capable of attracting the biological element
at least one array of microchannels (2) arranged between the reservoir (1) and the external medium (3) of the chip and allowing the chemoattractant compound to pass towards said medium and the biological element present in said medium to pass towards the reservoir (1), characterized in that each microchannel is in the form of a harpoon comprising at least two arrows (4) spaced longitudinally from one another and directed towards the reservoir, wherein each arrow comprises two branches (5) each having a free end (6) forming an acute angle of between 10° and 80°, and wherein each arrow comprises two openings (7) communicating with a longitudinal part of the microchannel and having a width of between 5 μm and 30 μm.

Preferably, the present invention concerns a microfluidic chip designed to be implanted in vivo to attract and trap and ultimately destroy a specific biological element, preferably a eukaryotic cell, and even more preferably a cancer cell.

The reservoir matrix containing the chemoattractant compound is made of a biocompatible material.

The chemoattractant compound in the reservoir matrix attracts the specific biological element to the chip in vivo by positive chemotaxis, with the biological element migrating towards the reservoir where the concentration of chemoattractant compound is highest. The array of microchannels allows the chemoattractant compound to diffuse from the reservoir to the chip's external environment, and the specific biological element to pass from the chip's external environment to the chip's interior, in the direction of the reservoir. Advantageously, the particular shape of each microchannel composing the array of microchannels, i.e., in the form of a harpoon comprising at least two arrows spaced longitudinally from one another and directed towards the reservoir, with each arrow comprising two branches each having a free end forming an acute angle of between 10° and 80°, and with each arrow comprising two openings communicating with a longitudinal part of the microchannel and having a width of between 5 μm and 30 μm, allows the passage of the biological element from the external environment within the microfluidic chip in the direction of the reservoir while preventing the passage of the biological element present in said chip in the direction of the external environment. In this way, the array of microchannels implemented in the chip according to the invention not only traps the biological element present in the chip, but also passively destroys it, since said element trapped within the chip is deprived of the conditions necessary for its survival.

In particular, when the biological element attracted and trapped in the microfluidic chip is a prokaryotic or eukaryotic cell, in the absence of the conditions necessary for its survival, the cell will be destroyed, notably by apoptosis after a few hours to a few days. Cellular debris, or apoptotic bodies, exit the chip via the array of microchannels and are discharged into the external environment. In this way, the microfluidic chip enables passive destruction of the biological element, without the need for one or more electrodes or energy consumption. The chip's operating capacity is continuously renewed thanks to the passive destruction of the biological element trapped within the chip, and also thanks to its passive release to the outside environment. The dimensions and geometry of the microchannels implemented in the chip according to the invention are particularly suitable for enabling the movement of eukaryotic cells which need a support to migrate, in particular by projecting their membranes forward and forming an actin-rich structure known as a lamellipod. In addition, the dimensions and geometry of the microchannels used in the chip according to the invention advantageously contribute to the release profile of the chemoattractant compound towards the external environment, in particular by creating a hydrodynamic resistance which limits the diffusion of the chemoattractant compound towards the outside and contributes to the creation and maintenance of a chemoattractant compound gradient. Finally, the dimensions and geometry of the microchannels used in the chip according to the invention ensure the unidirectional passage of a specific biological element within the chip from the external environment towards the chemoattractant compound reservoir, thus enabling the element to be trapped within the chip.

According to a preferred aspect, each microchannel comprises between 2 and 6 arrows, preferably between 3 and 5 arrows and even more preferably 3 arrows.

According to another preferred aspect, each free end of an arrow forms an angle of between 10° and 60°, preferably between 10° and 45°, more preferably between 10° and 30°, even more preferably an angle of 20°.

According to another preferred aspect, the two openings present on each arrow and communicating with the longitudinal part of the microchannel have a width of between 10 μm and 20 μm, preferably between 10 μm and 15 μm, even more preferably 10 μm.

In a particular embodiment of the invention, the two openings present on each arrow and communicating with the longitudinal part of the microchannel have a width of less than 10 μm. The advantage of this design is that it enhances the chip's ability to specifically target and attract cancer cells, which are the only eukaryotic cells capable of invaginating so as to pass through the aforementioned openings.

In particular, in the chip according to the invention, the two openings present on each arrow are located on the longitudinal axis of the microchannel and communicate with the longitudinal part of said microchannel.

In the microfluidic chip according to the invention, each arrow comprises a first “inlet” opening at the entrance to the arrow, through which the biological element enters, and a second “outlet” opening through which the biological element exits towards the reservoir comprising the chemoattractant compound. In other words, the present invention concerns a microfluidic chip in which each arrow (4) comprises a first opening through which the biological element enters and a second opening through which the biological element exits in the direction of the reservoir comprising the chemoattractant compound.

In the microfluidic chip according to the invention, each branch of the same arrow is symmetrical to the other branch along the longitudinal axis of the microchannel.

According to a particular aspect, the present invention concerns a microfluidic chip according to the first object, in which the free end (6) of each branch (5) is separated from the other by a distance of between 30 and 200 μm. Preferably, the free end (6) of each branch (5) is separated from the other by a distance of between 50 and 100 μm, more preferably by a distance of between 50 and 70 μm.

According to another particular aspect, the present invention relates to a microfluidic chip in which each arrow (4) is longitudinally spaced from the next by a distance of between 10 and 100 μm, preferably between 10 and 30 μm.

More particularly, the present invention relates to a microfluidic chip in which each arrow (4) comprises a first opening through which the biological element enters and a second opening through which the biological element exits towards the reservoir (1) and in which the second opening of one arrow is spaced longitudinally from the first opening of the following arrow by a distance of between 10 and 100 μm, preferably between 10 and 30 μm.

Preferably, the length formed by all the arrows on the same microchannel and the longitudinal space between each of them is between 100 μm and 500 μm, preferably between 200 μm and 250 μm.

According to a still more particular aspect, the present invention concerns a microfluidic chip in which each arrow (4) has a length of between 50 and 200 μm, preferably 50 μm, a height of between 10 and 50 μm, preferably 10 μm and a distance between each free end (6) of each branch (5) of between 30 and 200 μm, preferably 50 μm.

According to a preferred aspect of the invention, each arrow (4) has a length of 50 μm, a height of 10 μm and a distance between each free end (6) of each branch (5) of 50 μm.

Preferably, all the arrows present on the same microchannel have the same dimensions.

According to a still more particular aspect, the present invention concerns a microfluidic chip in which each microchannel has a length of between 100 and 500 μm, preferably between 200 and 300 μm, a width of between 30 and 200 μm, preferably between 50 and 100 μm, a height of between 5 and 50 μm, preferably between 20 and 30 μm.

Preferably, each microchannel in an array of microchannels is separated from the directly adjacent microchannel by a distance of between 50 and 400 μm, preferably between 100 and 200 μm.

The microchannels making up the array of microchannels of the microfluidic chip according to the present invention can be parallelepipedal, cylindrical, squamous, frustoconical or a mixture of these shapes.

Preferably, in the context of the present invention, all the microchannels included in an array of microchannels have the same shape and dimensions.

According to a particular embodiment of the present invention, each microchannel comprises 3 arrows, each free end (6) of an arrow (4) of which forms an angle of 20° and the two openings (7) present on each arrow have a width of between 10 μm and 20 μm. Preferably, the free end (6) of each leg (5) is separated from the other by a distance of between 50 and 100 μm. Even more preferably in this embodiment, each arrow (4) has a length of 50 μm, a height of 10 μm and a distance between each free end (6) of each branch (5) of 50 μm.

Even more preferably, each arrow is separated longitudinally from the next by a distance of between 10 and 30 μm.

Even more preferably, each microchannel has a length of between 100 and 500 μm, a width of between 30 and 200 μm and a height of 10 μm.

In particular, the present invention concerns a microfluidic chip in which said chip is devoid of electrodes between the reservoir (1) and the array of microchannels (2).

Advantageously, the absence of an electrode makes the chip easier to manufacture and implement, and also provides a chip that can passively attract, trap and ultimately destroy a specific biological element.

In the context of the present invention, the biological element is preferably a cancer cell, and in particular a cancer cell originating from a cancer or solid tumor. This cell migrates inside the chip by adhering to the support on which the array of microchannels is located.

Preferably, the array of microchannels of the microfluidic chip according to the present invention is located on the outer edge of said chip, i.e., in contact with the external environment, thus ensuring communication between said environment and the interior space of the chip.

The chip according to the present invention may comprise several arrays of microchannels, for example two arrays of microchannels.

The microfluidic chip according to the first object of the invention comprises a lower part (8) and an upper part (9), the reservoir (1) comprising the chemoattractant compound matrix and the array of microchannels (2) being able to be comprised independently of each other in the upper part (9) and/or the lower part (8) of said chip.

According to a particular embodiment, the microfluidic chip according to the first object of the invention comprises a lower part (8) comprising part of the reservoir (1), the array of microchannels (2) and an upper part (9) comprising part of the reservoir (1) and suitable for being arranged on the lower part (8), said parts being fixed to one another.

According to another particular embodiment, the microfluidic chip according to the present invention comprises a lower part (8) comprising part of the reservoir (1), and an upper part (9) comprising part of the reservoir (1) and the array of microchannels (2), said upper part being suitable for being arranged on the lower part (8), said parts being fixed to each other.

The upper part of the microfluidic chip can be placed on the lower part to form a cover, said parts being fixed together. The reservoir included in the lower part and in the upper part corresponds to one and the same reservoir, one part of which is located in the upper part of the chip and the other part in the lower part of the chip.

In the context of the present invention, the term “upper part suitable for being placed on the lower part to form a cover” means that the shape of the upper part is such that it adapts to that of the lower part on which it rests without hindering the functionality of each element making up the lower part, and makes it possible to form a cover closing the chip.

Advantageously and preferably, the upper and lower parts of the microfluidic chip are rounded so that the chip can be implanted in vivo without damaging the tissue.

Preferably, the chip is rounded in shape, with the upper and lower parts, for example, having a semi-oval or semi-spherical shape, so that when the upper part is placed on the lower part, the microfluidic chip is oval or spherical, respectively.

The size of the microfluidic chip according to the first object is suitable for in vivo implantation and is of the order of a few centimeters, preferably between 0.5 and 5 cm, more preferably between 1 and 3 cm, most preferably 1 cm.

More particularly, the present invention relates to a microfluidic chip in which the reservoir (1) and the array of microchannels (2) are annular in shape and in which the reservoir is located in the center of the chip.

As the name suggests, the annular shape corresponds to a ring. This particular configuration ensures radial diffusion of the chemoattractant compound contained in the reservoir towards the external medium, and homogeneous attraction of the biological element from said medium towards the reservoir, thanks to the central location of the reservoir and the annular shape of the array of microchannels. The lower and upper parts of the microfluidic chip can be attached to each other by any physical or chemical means suitable for in vivo use. An example of a physical or chemical means of attachment is a screw or glue, respectively, suitable for in vivo use of the chip, or a protruding element facing a hollow element in the lower and upper part to join them together.

Preferably in the context of the present invention, the upper part comprises one or more openings through which one or more screws can be inserted and the lower part comprises one or more nuts suitable for receiving said screw or screws.

Even more preferably, the upper part of the microfluidic chip according to the invention comprises a central opening through which a screw (17) can be inserted, and the lower part comprises a central nut suitable for receiving said screw. In this preferred embodiment, the annular reservoir is arranged around the central opening present on the upper part and the central nut present on the lower part of the chip.

In particular, the microfluidic chip comprises one or more gaskets (10) for sealing the chip, said gaskets being arranged between the lower part and the upper part above the array of microchannel.

The term “chip-tight seal” refers to the seal's ability to prevent fluids that may be present in the external environment, such as blood, from entering the chip, and to prevent liquids and materials present inside the chip from escaping to the external environment anywhere other than via the array of microchannels. The position of the seal(s) above the array of microchannels ensures that neither the diffusion of the chemoattractant compound into the external environment nor the passage of the specific cell present in the said medium within the chip, towards the reservoir, is impeded. Advantageously, the seals create a support zone between the upper and lower parts of the chip.

In particular, the present invention concerns a microfluidic chip in which the upper part (9) and/or the lower part (8) comprise an annular cavity i) (8) suitable for receiving the matrix comprising the chemoattractant compound, and an annular cavity ii) (10) disposed between the reservoir (1) and the array of microchannels (2), suitable for receiving a liquid in which the chemoattractant compound is suitable for diffusion.

More particularly, the present invention concerns a microfluidic chip in which the upper part (6) and/or the lower part (5) comprise an annular cavity i) (11) able to receive the matrix comprising the chemoattractant compound via one or more openings (12) communicating with the external environment (3) and opening into said cavity, and an annular cavity ii) (13) located between the reservoir (1) and the array of microchannels (2), suitable for receiving a liquid into which the chemoattractant compound is able to diffuse, via one or more openings (14) communicating with the external medium (3) and opening into said cavity.

More particularly, the present invention concerns a microfluidic chip in which the upper part (9) and the lower part (8) comprise an annular cavity i) (11) able to receive the matrix comprising the chemoattractant compound via one or more openings (12) communicating with the external environment (3) and opening into said cavity, and an annular cavity ii) (13) located between the reservoir (1) and the array of microchannels (2), suitable for receiving a liquid into which the chemoattractant compound is able to diffuse, via one or more openings (14) communicating with the external medium (3) and opening into said cavity.

Preferably, the chemoattractant compound is added to the reservoir matrix before it is added to the annular cavity i). Alternatively, the chemoattractant compound can be added to the annular cavity i) before or after the reservoir matrix is added to the same cavity.

The matrix comprising the chemoattractant compound and the liquid into which the chemoattractant compound is able to diffuse are added respectively to the annular cavity i) and to the annular cavity ii) via said openings (12, 14), preferably after the upper part has been placed on the lower part.

The liquid into which the chemoattractant compound is able to diffuse is preferably an aqueous solution such as physiological saline or a physiological buffer solution such as an aqueous solution comprising phosphate-buffered saline (PBS).

The sealing of each of the cavities after said additions is ensured by closing the openings via gaskets, for example in PDMS or any other suitable material, said gaskets being located at the outlet of these openings (12, 14) and communicating with the external environment.

Preferably, the annular cavity i) is included in the upper and lower part of the chip, the opening or openings leading into this cavity being located in the lower part of the chip, and the annular cavity ii) and the opening or openings leading into this cavity are included in the upper part of the chip.

The chip according to the present invention is made of biocompatible material. In particular, the present invention relates to a microfluidic chip in which the upper part, the lower part and the matrix comprising the chemoattractant compound are composed of a biocompatible material.

More precisely, the upper part and the lower part as such as well as the elements they comprise are made of a biocompatible material. Even more precisely, the upper part, the lower part, the reservoir including the matrix comprising the chemoattractant compound, the array of microchannels, and the joints are made of a biocompatible material.

The term “biocompatible material” or “biomaterial” refers to a material that does not interfere with or degrade the biological environment in which it is used, even when in direct or indirect, brief or prolonged contact with the internal tissues and fluids of a human or animal body. Examples of biocompatible materials that can be used in the context of the present invention include, but are not limited to, glass, ceramics such as alumina, zirconia and hydroxyapatite, metals and metal alloys such as titanium and platinum, polymers of natural origin such as collagen, agarose, chitosan, carrageenan, xanthan and alginate, or degradable synthetic polymers such as polyesters and polyanhydrides, or non-degradable polymers such as polyurethanes, cellulose and its derivatives, vinyl polymers. Preferably in the context of the present invention, polymers of synthetic origin are PEEK (polyetheretherketone) or PDMS (polydimethylsiloxane).

Preferably, the upper part, the lower part and the array of microchannels are, independently of each other, made of polymers of synthetic origin such as polydimethylsiloxane (PDMS) or polyetheretherketone (PEEK). Even more preferably, the upper and lower parts are made of polyetheretherketone (PEEK).

Preferably, the array of microchannels is made of polyetheretherketone (PEEK).

Preferably, the reservoir and in particular the matrix comprising the chemoattractant compound is made of collagen and/or alginate, still more preferably said reservoir, in particular said matrix is made of alginate.

In a particular and preferred way, the upper part, the lower part and the array of microchannels of the chip according to the invention are made of polyetheretherketone (PEEK), the reservoir of chemoattractant compound is made of alginate.

According to a particular aspect, the present invention relates to a microfluidic chip in which the matrix comprising the chemoattractant compound is composed of a biocompatible material, preferably a cross-linked polymer. Preferably, the cross-linked polymer making up the matrix is a polymer of natural origin and in particular collagen and/or alginate, preferably alginate.

Polymer crosslinking refers to the formation of one or more three-dimensional networks from linear or branched polymers, by chemical and/or physical means. Those skilled in the art know how to induce cross-linking of polymers, depending on the polymers in question. For example, cross-linking can be achieved by heating and/or using a cross-linking agent. A “cross-linked” polymer is one in which some of its chains are linked together by strong or weak bonds.

By way of example, collagen can be cross-linked using cross-linking agents such as ammonia gas, oxidized sugars or aldehydes at room temperature, and alginate can be cross-linked in a calcium chloride bath at room temperature.

Even more preferably, the biocompatible material making up the matrix comprising the chemoattractant compound is alginate crosslinked in a calcium chloride bath, preferably at room temperature.

Cross-linking of the biocompatible material making up the matrix comprising the chemoattractant compound advantageously enables prolonged release of this compound. “Prolonged release” refers to controlled and continuous release kinetics of the chemoattractant compound over a period of time. Preferably in the context of the present invention, release of the chemoattractant compound takes place between 3 days and 6 months, preferably between 15 days and 3 months.

According to a particular aspect, the present invention concerns a microfluidic chip in which the mass percentage of chemoattractant compound/matrix in the reservoir (1) is between 0.1 and 20%, preferably between 0.5 and 10% and even more preferably between 0.5 and 5%.

Those skilled in the art will be able to determine the content of chemoattractant compound in the reservoir according to the context of use of the chip, the specific biological element of interest, the desired release time of the chemoattractant compound and the biomaterial making up the reservoir matrix.

When the specific biological element is a eukaryotic cell, preferably a cancer cell, the chemoattractant compound included in the microfluidic chip reservoir and in particular in the reservoir matrix, is preferably selected from chemokines such as CXCL12, also known as stromal cell-derived factor 1 (SDF-1), CCL5, CCL2, CCL3, CCL7, CCL19, CCL21, CCL22, CCL25, CXCL1, CXCL5, CXCL6, CXCL8, CX3CL1, growth factors and transforming growth factors such as TGF alpha or beta (transforming growth factor alpha or beta), FGF (fibroblast growth factor alpha), EGF (epidermal growth factor alpha), betacellulin, amphiregulin, heregulin, HBEGF, PDGF (platelet-derived growth factor), VEGF (vascular endothelial growth factor).

More particularly, the present invention relates to a microfluidic chip in which the chemoattractant compound included in the chip reservoir is selected from at least one of the following compounds: CXCL12, CCL5, CCL2, CCL3, CCL7, CCL19, CCL21, CCL22, CCL25, CXCL1, CXCL5, CXCL6, CXCL8, CX3CL1, TGF alpha, TGF beta, FGF, PDGF, EGF, VEGF.

According to another aspect, when the specific biological element is a prokaryotic cell, preferably a bacterium, the chemoattractant included in the reservoir of the microfluidic chip and in particular in the reservoir matrix, is preferably chosen from carbohydrate molecules.

The chemoattractant compound can be used alone or in combination with one or more of the other chemoattractant compounds mentioned above and/or with other compounds capable of directly or indirectly improving the ability of said chemoattractant compound to attract a specific biological element, such as a eukaryotic cell and in particular a cancer cell, such as carbohydrate (glucose) and/or lipid (fatty acid) molecules which provide the energy required (energy supplied in the form of ATP after degradation of glucose or fatty acids) for the survival of the biological element, in particular a eukaryotic cell and in particular a cancer cell. Other molecules such as oxygen can be used in association with chemoattractants. Oxygen can be transported by hemoglobin or synthetic hemoglobins. Oxygen is an essential molecule for the survival and proliferation of cells, particularly cancer cells. Preferably, the chemoattractant compound is used in combination with one or more carbohydrate (glucose) and/or lipid (fatty acid) molecules.

The person skilled in the art will choose the chemoattractant compound(s) according to the specific biological element targeted.

To this end, when the biological element is a eukaryotic cell, an analysis of the membrane receptors expressed by the targeted cell must be carried out upstream to ensure the specificity of the chemoattractant compound(s) chosen.

For example, when the specific biological element is a cancer cell expressing the CXCR4 transmembrane receptor, such as a human breast cancer cell, the chemoattractant compound chosen is stromal cell-derived factor 1 (SDF-1).

Similarly, when the specific biological element is a lung cancer cell, the chemoattractant chosen is epidermal growth factor (EGF) or transforming growth factor alpha (TGF alpha).

A second object of the invention concerns the use of the microfluidic chip according to the first object of the invention to attract and trap a specific biological element.

More particularly, the present invention relates to the use of the microfluidic chip according to the first object of the invention or a method for attracting and trapping a specific biological element in vivo, said chip being implanted in vivo.

More precisely, the present invention relates to the use of the microfluidic chip according to the first object of the invention or a method for treating or preventing the proliferation and dissemination of a specific biological element in a subject, such as a prokaryotic or eukaryotic cell in which the chip is implanted in vivo in a subject.

According to one aspect, the present invention relates to the use of the microfluidic chip according to the first object of the invention or a method for treating or preventing an infection caused by a prokaryotic cell such as a bacterium in which the chip is implanted in vivo in a subject.

According to a preferred aspect, the present invention relates to the use of the microfluidic chip according to the first object of the invention or a method for treating or preventing proliferation and dissemination of a eukaryotic cell and in particular a cancer cell, preferably after resection of a solid tumor in a subject, wherein the chip is implanted in vivo in a subject.

More specifically, the present invention relates to the use of the microfluidic chip according to the first object of the invention or to a method for preventing the risks of local cancer recurrence and/or metastatic cancer development in a subject, in which the chip is implanted in vivo in a subject.

In these uses and methods, the microfluidic chip is implanted at a distance of 0.1 to 20 cm, preferably 1 to 10 cm, more preferably at a distance of 5 cm from the resection zone of a solid tumor or the focus of bacterial infection in a subject. The microfluidic chip is preferably implanted in the resection area as soon as the solid tumor is removed, preferably immediately after resection of the tumor.

The term “context of use of the chip” refers here to the type of specific biological element targeted, i.e., the type of bacterial infection when said element is a bacterium, or the type of cancer cell targeted and in particular the type of solid tumor removed and the stage of said tumor when said element is a cancer cell.

In the context of the above uses, the microfluidic chip can be used alone or in combination with the simultaneous or sequential administration of other drug compounds such as anti-cancer compounds, in particular chemotherapeutic and/or hormone therapy and/or immunotherapy and/or targeted therapy and/or radiotherapy when the specific biological element is a cancer cell.

Finally, the present invention also relates to a microfluidic chip according to the first object for use in attracting and destroying a specific biological element in vivo, said chip being implanted in vivo, in accordance with the aforementioned implementation conditions.

More precisely, the present invention relates to a microfluidic chip according to the first object for use in treating or preventing the proliferation and dissemination of a specific biological element in a subject, such as a prokaryotic or eukaryotic cell, wherein the chip is implanted in vivo in a subject in accordance with the aforementioned implementation conditions.

According to one aspect, the present invention relates to a microfluidic chip according to the first object for use in treating or preventing an infection caused by a prokaryotic cell such as a bacterium, wherein the chip is implanted in vivo in a subject.

According to a preferred aspect, the present invention relates to a microfluidic chip according to the first object for use in treating or preventing proliferation and dissemination of a eukaryotic cell and in particular a cancer cell, preferably after resection of a solid tumor in a subject, wherein the chip is implanted in vivo in a subject in accordance with the aforementioned implementation conditions.

Even more preferably, the present invention relates to a microfluidic chip according to the first object for use in preventing the risks of local cancer recurrence and/or metastatic cancer development in a subject, wherein the chip is implanted in vivo in a subject in accordance with the aforementioned implementation conditions.

The duration of use of the microfluidic chip in vivo can be between 2 and 12 months. After this period, a new chip can be implanted.

In the context of these uses and methods, when the specific biological element is a eukaryotic cell and in particular a cancer cell, said cell preferably comes from a cancer of the breast, lungs, prostate, bladder, salivary glands, skin, intestine, colon-rectum, thyroid, cervix, endometrium and ovaries, or from a cancer of the ovaries, salivary glands, skin, intestine, colon-rectum, thyroid, cervix, endometrium and ovaries, lip-mouth-larynx cancer, kidney, liver, brain, testicles, pancreas, preferably breast cancer. These examples of solid tumors are not exhaustive.

The invention will be further illustrated by the following figures and examples. However, these examples and figures must in no way be interpreted as limiting the scope of the invention.

### EXAMPLES

### Example 1: Manufacture of Microfluidic Chip for In Vitro Concept Study

To carry out the in vitro proof-of-concept, a microfluidic chip containing a reservoir at its center, in which a chemoattractant is placed with an array of microchannels in the form of harpoons, in accordance with the present invention has been manufactured.

a) Manufacture of the SU-8 Photosensitive Epoxy Resin Mold from MicroChemicals

A silicon wafer (diameter 76.2 mm) is prepared in a “piranha” solution, followed by dehumidification for 15 to 150° C. SU-8 resin is deposited on the silicon plate by centrifugal coating (3000 rpm, 30 s) to obtain the required thickness (10 μm or 100 μm depending on the type used SU-8 2010 or SU-8 2100). Solvent evaporation is achieved with very light heating and cooling ramps to minimize mechanical stress in the resin (5° C./min). The sample is subjected to a 365 nm UV exposure of 125 mJ/cm2 for a thickness of 10 μm, and 250 mJ/cm2 for a thickness of 100 μm. The plate is then subjected to a post exposure bake (PEB) (4 min at 95° C. for a thickness of 10 μm and 30 min at 95° C. for a thickness of 100 μm). Finally, the uncrosslinked parts of the SU-8 resin are diluted in a solvent (“SU-8 developer”, mainly composed of PGMEA (propylene glycol monomethyl ether acetate)). At the end of this stage, the patterns used to structure the harpoon-shaped microchannels remain on the silicon wafer.

### b) Microchannel Structuring and Final Chip Assembly

Following mold manufacture, a silicone polydimethylsiloxane (PDMS) polymer is prepared by mixing it with its curing agent (Sylgard™184 silicone elastomer kit from Dow, ratio 1:10). Bubbles formed during mixing are removed using a desiccator and vacuum pump. Once the PDMS has been degassed, it is poured onto the SU-8 mold placed in a Petri dish, then placed in an oven at 80° C. for at least 2 hours to complete the crosslinking process. After cooling, the PDMS is peeled off, then cut with a circular blade to obtain the cylindrical shapes of the devices. These are then pierced with a punch to form the central well that will contain the chemoattractants. The final step is to bond the PDMS to a glass substrate to encapsulate the channels. This is achieved by activating the surface to transform the Si—CH₃ function of PDMS into Si—OH using an O₂ or air plasma generator. On contact with the glass (SiO₂), a permanent Si—O—Si covalent bond is created.

### c) Manufacture of Release Matrix

A 3% (w/v) aqueous alginate solution was deposited in a porous mold in the shape of the chip reservoir. The mold was immersed in a calcium chloride cross-linking bath for 24 hours. After 3 washes with miliQ water, the matrix was frozen at −20° C. and freeze-dried.

The matrix is then gently placed in the chip.

### Example 2: Implementation of the In Vitro Microfluidic Chip

### A) Cell Attraction Capacity Inside the Chip

The microfluidic chip was implemented in vitro to test its ability to attract MDA-MB-231 breast cancer cells, which are epithelial cells from mammary tumors. The SDF-1 “stromal cell-derived factor” is the chemoattractant compound capable of attracting MDA-MB-231 cells.

1) MDA-MB-231 cells stably transfected with green fluorescent protein (GFP) are trypsinized at D0. 20,000 cells are seeded in a 35 mm petri dish containing the in vitro microfluidic chip. Cells are cultured in Dulbecco's Modified Eagle Medium/Nutrient Mixture F-12 (DMEM F12) 1% fetal calf serum (SVF)+1% antibiotic (streptomycin, penicillin).

The central reservoir of the in vitro microfluidic chip is loaded with either 50 μL DMEM F12+1% SVF (fetal calf serum)+1% antibiotic (streptomycin, penicillin) (negative control) or 50 μL SDF-1 alpha at 100 ng/ml in 0.1% BSA in DMEM F12+1% antibiotic (streptomycin, penicillin). After 2, 4 and 7 days of culture in a 5% CO2 incubator at 95% humidity, photographs were taken using an inverted fluorescence microscope, and the cells on the chip were counted after 4 and 7 days. The results are shown in Table 1 below:

TABLE 1
Cell counting using an inverted fluorescence microscope

| Measurement Day | 4 | 7 |
|---|---|---|
| Negative Control | 183 | 154 |
| SDF-1 alpha 100 ng/ml | 185 | 850 |

In the presence of the chemoattractant SDF-1 alpha, cells move from the outside to the inside of the chip, and in particular to the central reservoir. On day 7 in the chip containing the chemoattractant, there were 850 cells in the chip, distributed throughout the chip and in particular in the central reservoir. On the other hand, on day 7 in the chip not containing the chemoattractant, i.e., the negative control chip, there were 154 cells on the chip, located exclusively at the exit of the microchannels inside the chip.

In another experiment, under the same experimental conditions as above, an in vitro microfluidic chip equipped with harpoon microchannels, containing the chemoattractant SDF-1 alpha at 100 ng/ml in its reservoir, was placed in an incubator under an inverted microscope equipped with an λ10 objective and a camera enabling cells to be monitored after 5 to 6 days of culture.

The number of cells passing through the chip after 5 to 6 days of culture on a portion of the chip corresponding to 7 microchannels was quantified, as well as the number of cells exiting the chip after 5 to 6 days of culture. This number was then extrapolated to 200 microchannels, corresponding to the average number of microchannels present in the array of microchannels of a chip according to the invention.

The results obtained are presented in Table 2 below.

TABLE 2
Cell counting using an inverted microscope
coupled to a camera with an X10 objective

| | On a portion of the | Extrapolated to a |
|---|---|---|
| | chip with 7 | chip with 200 |
| | microchannels | microchannels |
| Number of cells passing | 18 | 514 |
| through the chip within | | |
| 5 to 6 days of culture | | |
| Number of cells released | 0 | 0 |
| from the chip within 5 | | |
| to 6 days of culture | | |

Cells enter the harpoon-shaped microchannels and, once inside, are unable to turn back. The cells are thus trapped inside the chip and end up dying inside either the microchannels or the central reservoir (see FIG. 2).

1. A microfluidic chip for attracting and trapping a biological element, said chip comprising:
a reservoir (1) having a matrix comprising a chemoattractant compound configured to attract the biological element, and
at least one array of microchannels (2) arranged between the reservoir (1) and the external medium (3) of the chip and enabling the chemoattractant compound to pass towards said medium and the biological element present in said medium to pass towards the reservoir (1),
wherein each microchannel is in the form of a harpoon comprising at least two arrows (4) spaced apart longitudinally and directed towards the reservoir,
wherein each arrow comprises two branches (5) each having a free end forming an acute angle of between 10° and 80°, and
wherein each arrow comprises two openings (7) communicating with a longitudinal part of the microchannel and having a width of between 5 μm and 30 μm.

2. The microfluidic chip according to claim 1, wherein the free end (6) of each branch (5) is separated from the other by a distance of between 30 and 200 μm.

3. The microfluidic chip according to claim 1, wherein each arrow (4) comprises a first opening through which the biological element enters and a second opening through which the biological element exits towards the reservoir (1), and wherein the second opening of one arrow is spaced longitudinally from the first opening of the following arrow by a distance of between 10 and 100 μm.

4. The microfluidic chip according to claim 1, wherein each arrow (4) has a length of between 50 and 200 μm, a height of between 10 and 50 μm and a distance between each free end (6) of each branch (5) of between 30 and 200 μm.

5. The microfluidic chip according to claim 1, wherein each microchannel has a length of between 100 and 500 μm, a width of between 30 and 200 μm, a height of between 5 and 50 μm.

6. The microfluidic chip according to claim 1, wherein the chip is devoid of electrodes between the reservoir (1) and the array of microchannels (2).

7. The microfluidic chip according to claim 1, comprising a lower part (8) comprising part of the reservoir (1) and an upper part (9) comprising part of the reservoir (1) and the array of microchannels (2), said upper part being suitable for being arranged on the lower part (8), said parts being fixed to one another.

8. The microfluidic chip according to claim 1, wherein the reservoir (1) and the array of microchannels (2) are annular in shape, and wherein the reservoir is located in the center of the chip.

9. The microfluidic chip according to claim 7, wherein the upper part (9), the lower part (8) and the matrix comprising the chemoattractant compound are composed of a biocompatible material.

10. The microfluidic chip according to claim 1, wherein the mass percentage of chemoattractant compound/matrix in the reservoir (1) is between 0.1 and 20%, preferably between 0.5 and 10% and even more preferably between 0.5 and 5%.

11. The microfluidic chip according to claim 1, wherein the specific biological element is a eukaryotic cell.

12. The microfluidic chip according to claim 11, wherein the eukaryotic cell is a cancer cell.
